(19) United States
(12) Patent Application Publication
(10) Pub. No.: US 2004/0093037 A1
(43) Pub. Date: May 13, 2004

(54) Discrimination between ventricular tachycardia and ventricular fibrillation in an active implantable medical device of the defibrillator, cardiovertor and/or antitachycardia pacemaker type

(76) Inventor: Christine Henry, Paris (FR)

Correspondence Address: Robert M. Isackson, Orrick, Herrington & Sutcliffe LLP, 666 Fifth Avenue, New York, NY, 10103, US

(21) Appl. No.: 10/654,276

(22) Filed: Sep. 3, 2003

(30) Foreign Application Priority Data
Sep. 4, 2002 (FR) 02 10912

(51) Int. Cl. A61N 1/365

(52) U.S. Cl. 607/14

(57) ABSTRACT

Discrimination between ventricular tachycardia and ventricular fibrillation in an active implantable medical device of the defibrillator, cardiovertor and/or antitachycardia pacemaker type. This device compares the ventricular rate or frequency with a first threshold (TDI) of detection of ventricular tachycardia, a second threshold (FDI) of detection of ventricular fibrillations, and a third threshold (F/TDI) intermediate the first and second thresholds. When the frequency is included between the third threshold (F/TDI) and second threshold (FDI), an additional discrimination is operated to discriminate “super-rapid” ventricular tachycardia and ventricular fibrillation, based on an analysis of the stability of the ventricular frequency RR intervals. A shock therapy is applied only if the determined ventricular frequency is recognized as unstable. If not unstable, an antitachycardia pacing stimulation sequence is applied. When the determined ventricular frequency is included between the first threshold (TDI) and third threshold (F/TDI), the device normally continues the analysis of the cardiac rhythm based on other criteria to discriminate ventricular tachycardia by known criteria.

### FIELD OF THE INVENTION

The present invention relates to active implantable medical devices (within the meaning of the Jun. 20, 1990, directive 90/385/CEE of the Council of the European Communities), and more particularly to the devices commonly called implantable “defibrillators” or “cardiovertors,” it being understood that the invention is equally as well applicable to implantable defibrillator/cardiovertor/pacemaker and implantable defibrillator/pacemaker devices.

### BACKGROUND OF THE INVENTION

Implantable defibrillators and cardiovertors are among the class of devices that diagnose certain tachyarrhythmia conditions and deliver to the heart a stimulation therapy, specifically defibrillation or cardioversion “shocks” (i.e., stimulation pulses of relatively high energy, notably exceeding the energy typically provided for simple stimulation of the cardiac rhythm). Some of these devices also incorporate a stimulation therapy mode called “ATP” (AntiTachycardia Pacing) which involves applying a programmed series of high frequency stimulation pulses at an energy level below the shock energy and more typically near to or at the simple stimulation energy level.

The decision whether to apply an antitachycardia stimulation therapy, and the choice of which stimulation therapy to apply (shock or ATP) is made by application of an algorithm for the detection and classification of the various tachyarrhythmia. The algorithm typically employs several predetermined criteria, mainly the determined ventricular frequency (or frequency rate, the terms being used interchangeably, but also the stability of the detected intervals separating ventricular events, the stability of atrio-ventricular conduction, and the mode of starting of tachycardia, etc. Reference in this regard is made to, for example, EP-A-0 626 182 and its corresponding U.S. Pat. No. 5,462,060, and to EP-A-0 838 235 and its corresponding U.S. Pat. No. 5,868,793, commonly assigned herewith to ELA Médical, Montrouge France, for a description of such an algorithm and preselected criteria.

If one puts aside those tachyarrhythmia for which ventricular therapies are not suitable, in particular sinusal tachycardia (ST) or supra-ventricular tachycardia (SVT) that are atrial in origin and for which a shock applied to the ventricle would be ineffective, and perhaps even noxious from a hemodynamic point of view, it is important that the algorithm also performs a discrimination between a fast ventricular tachycardia (VT) and a ventricular fibrillation (VF). This is because these two forms of tachycardia are treated differently. In the event of a proven ventricular fibrillation, or of an unstable fast polymorphic VT, it is important to apply a shock therapy as soon as possible, as it is the only solution that is reasonably likely to terminate such a tachyarrhythmia condition. On the other hand, in the event of a fast or slow monomorphic ventricular tachycardia, it is neither a priori systematically necessary nor is it useful to apply a shock. Instead, an ATP stimulation therapy can be applied in the first instance that can be effective to revert the VT and thereby avoid the application of a shock, a more painful event.

The algorithms for the classification of tachyarrhythmia operate according to several criteria, of which the first is that of the ventricular frequency or rate. If a finer analysis is necessary, the algorithm further evaluates the stability of RR intervals (ventricular intervals), the stability of the PR intervals (i.e., the atrio-ventricular association), the presence or not of an abrupt acceleration of the cardiac rate and the origin, ventricular or atrial, of this acceleration. The first criterion, the ventricular frequency, makes it possible in particular to distinguish three situations according to whether the frequency is more or less high (or, in an equivalent respect, the corresponding interval RR is more or less short relative to a threshold (limit value)). These situations are illustrated on FIG. 1, which represents the various ranges of ventricular frequency with the corresponding diagnoses and actions, in accordance with the prior art. The three situations are:

1. The ventricular frequency is below a given threshold, called the “frequency of detection of VT” or “threshold TDI” (Tachycardia Detection Interval), for example, about 140 bpm. The algorithm considers this to be a slow rate that is not pathological and never justifies the application of a stimulation therapy.

2. The ventricular frequency is between the frequency of detection of VT, typically 140 bpm, and another given threshold that is at a higher frequency, known as “frequency of detection of FV” or “threshold FDI” (Fibrillation Detection Interval), typically 200 bpm. The algorithm considers that in this range there is a “suspicion of VT” and carries out a more thorough analysis of detected cardiac events, implementing criteria other than the ventricular frequency. More precisely, the algorithm seeks to determine the type of arrhythmia disorder and to decide whether it is necessary to apply a stimulation therapy, and, if so, what therapy (shock or ATP).

3. The ventricular frequency is higher than the frequency of detection of FV, typically 200 bpm. The algorithm considers that the application of shock therapy is in any event necessary, and to be delivered without delay.

A difficulty lies, however, in the choice of the level at which to set these thresholds, and in particular, the threshold FDI. This selection is a critical point because it makes it possible to discriminate the VF from the VT. This threshold value being one that is programmed by the clinician, there is a tendency to program the threshold of detection of the VF (threshold FDI) to a relatively high value, typically higher than 220 bpm, so that the majority of the VT can benefit from the ATP stimulation therapy, which is programmable to be applied only in the zone of a determined VT. The effectiveness of the ATP stimulation therapy on a fast monomorphic VT (220 to 240 bpm) is indeed well documented and, as the ATP is a much less painful stimulation therapy for the patient, it appears preferable to treat these pathologies by ATP rather than by a shock.

But the detection of a VF can be hindered by setting the threshold FDI at too high a level. Indeed, in the event of VF the cardiac signal is unstable and its coupling can sometimes be shorter than the duration of the absolute period refractory of the device. This can result in detecting this cardiac rate occurring at 2:1 association, which is undesirable. In addition, certain spontaneous cardiac waves can be of too low an amplitude for the established detection threshold and consequently they can be under-detected, i.e., the detection sensitivity is too low to detect events.

Under these conditions, and according to the functions of the tachycardia detection algorithm in the defibrillators such as those described in the abovementioned patents, it can suffice that three out of eight cycles presenting a coupling interval having a duration higher than the interval of detection of fibrillation (interval FDI), whereupon the VF is seen as in zone of tachycardia, and thus leads to a false negative in the diagnosis. This tachycardia being unstable, the prior art algorithm consequently produces a diagnosis of ST or SVT (because of interval RR instability), leading to an inhibition of any therapy, and thus to a delay in the application of a defibrillation shock, which would be an appropriate therapy.

### OBJECTS AND SUMMARY OF THE INVENTION

It is therefore, an object of the present invention to mitigate the aforementioned situation by proposing a device which authorizes the programming of threshold of detection of ventricular fibrillations at a relatively high level, while allowing discrimination, without delay, between a fast VT and a VF likely to occur in this frequency range. Advantageously, the treatment could be thus adapted as well as possible: immediate delivery of a shock in the event of VF, and stimulation therapy without shock by ATP in the event of fast VT.

Broadly, the present invention proposes an improved defibrillator or cardiovertor of the known type as described, for example, by the EP-A-0 838 235 and U.S. Pat. No. 5,868,793 abovementioned, and including: means for collecting (sensing) ventricular and atrial cardiac activity (i.e., spontaneous cardiac activity); means for delivering an antitachycardia stimulation therapy selected from among a defibrillation shock, a cardioversion shock and an antitachycardia pacing sequence (“ATP”); and means for discriminating ventricular arrhythmias that are able to analyze the detected ventricular rhythm in relation to a plurality of criteria including the ventricular frequency, which is compared with a plurality of thresholds including a first threshold of detection of ventricular tachycardia and a second threshold of detection of ventricular fibrillations. The discriminating means further operates to inhibit an application of the stimulation therapies of shock and ATP when the determined ventricular frequency is lower than the first threshold, declare a suspicion of a ventricular tachycardia and continue the analysis of the detected cardiac rhythm with respect to the aforementioned criteria when the ventricular frequency is between the first threshold and the second threshold, and control the application of a shock therapy when the determined frequency rate is higher than the second threshold.

In a manner characteristic of the invention, the discriminating means is modified and improved to be able to compare the ventricular frequency with a third threshold that is selected to be between the first threshold and the second threshold, and thereby be able to suspect a ventricular tachycardia and to continue the analysis of the ventricular rhythm on the aforementioned criteria only when the ventricular frequency is included between the first threshold and the third threshold, and to operate an additional discrimination between ventricular tachycardia and ventricular fibrillation when the detected ventricular frequency is between the third threshold and the second threshold.

The third threshold is typically selected from between 190 and 210 bpm, for a second threshold that is selected from between 230 and 250 bpm. In a preferred embodiment, the second and third thresholds are selected with an interval separating them of between 20 and 50 bpm.

Advantageously, the discriminating means operate the aforementioned additional discrimination between ventricular tachycardia and ventricular fibrillation by an analysis of the stability of the ventricular rhythm, in particular by employing a statistical analysis of RR intervals (i.e., the instantaneous detected ventricular frequencies over a time period, preferably over a sliding window of a number of cardiac cycles). More preferably, the analysis includes the establishment of a histogram of intervals RR, the search for a peak of stability in the histogram, and the evaluation of the proportion of intervals included in this peak of stability.

After the aforementioned additional discrimination, the discriminating means can in particular command an application of a shock therapy when the ventricular rhythm is recognized as unstable, and command an application of an antitachycardia pacing therapy when this rhythm is recognized as stable.

### BRIEF DESCRIPTION OF THE DRAWINGS

Further benefits, features and characteristics of the present invention will become apparent to a person of ordinary skill in the art in view of the following detailed description of a preferred embodiment of the invention, made with reference to the annexed drawings, in which:

FIG. 1 illustrates the various frequency ranges taken into account by the algorithms of discrimination of prior art, with the corresponding diagnoses and actions; and

FIG. 2 illustrates the various frequency ranges taken into account by the algorithm of discrimination according to the invention, with the corresponding diagnoses and actions.

### DETAILED DESCRIPTION OF THE INVENTION

The present invention can be preferably implemented starting from the available algorithm known and described in the EP-A-0 626 182 and EP-A-0 838 235 and their respective above-mentioned corresponding U.S. Patents, which algorithm is used by the commercial models of DEFENDER™ and ALTO™ defibrillators manufactured by ELA Médical, to operate the detection and the classification of the various tachyarrhythmia according to various criteria.

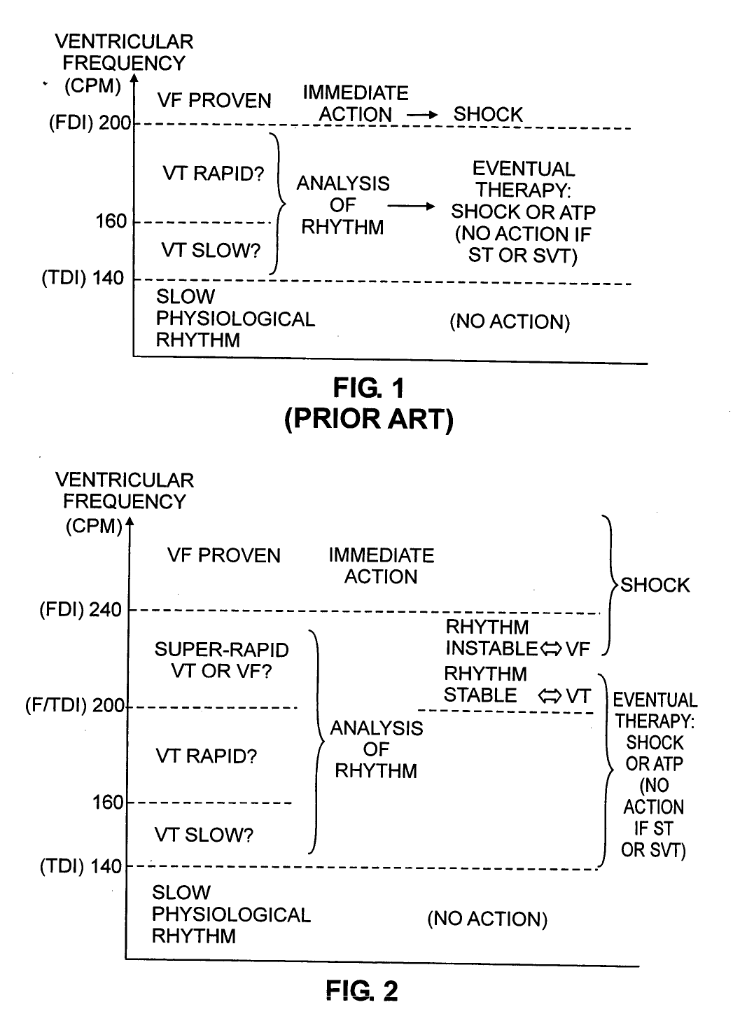

With reference to FIG. 1, in accordance with the prior art, this known algorithm makes it possible in particular to detect and confirm the occurrence of VT by an analysis of the cardiac rhythm. The analysis is implemented as soon as the ventricular frequency of the detected cardiac rhythm exceeds a programmed frequency (“frequency of detection of VT” or “threshold TDI”). It is in particular possible to discriminate, between various disorders, those which authorize the application of an antitachycardia therapy, i.e., a proven slow or fast VT, VF, and in addition those of non-ventricular origin for which any therapy of this type must be inhibited (i.e., supra-ventricular tachycardia (“SVT”), sinusal tachycardia (“ST”) and similar disorders). One will be able to refer to the above mentioned patents for further details, which U.S. Pat. Nos. 5,462,060 and 5,868,793 are incorporated herein by referenced in their entirety.

FIG. 2 illustrates the various cases taken into account by the improved algorithm of discrimination according to the present invention.

The starting point of the invention lies in setting threshold FDI at a relatively high level (typically 240 bpm) and creating below threshold FDI a particular detection zone for the range of frequencies (typically 200 to 240 bpm) where the algorithm is susceptible to detect VF as well as VT (as one will call “super-rapid VT”), in order to operate in this new zone a specific discrimination, that is much faster and is based on criteria different from those of an analysis of VT operated in accordance with the prior art.

The new threshold corresponding, indicated hereinafter as “threshold F/TDI” is established at a frequency that is lower than that of threshold FDI, for example, selecting a threshold F/TDI of 200 bpm for a threshold FDI of 240 bpm. The algorithm operates to compare with the various thresholds the frequency value of the detected ventricular rhythm:

1. For a ventricular frequency lower than threshold TDI: the detected cardiac rhythm is considered to be physiological, not justifying the application of a stimulation therapy;

2. For a ventricular frequency that is between threshold TDI and threshold F/TDI: there is a suspicion of VT, slow or fast, an implementation of a thorough analysis of the cardiac rhythm according to known discrimination criteria, for example, those described in the above-mentioned patents;

3. For a ventricular frequency that is higher than threshold FDI: the rate is considered immediately as revealing of a VF and a shock is applied without delay; and

4. For a ventricular frequency that is between threshold F/TDI and threshold FDI: an additional analysis is carried out, according to the present invention, in order to discriminate between VF and super-rapid VT.

This additional analysis is preferably an analysis of the stability of the ventricular rhythm, (e.g., the stability of intervals RR), for example, in the manner described in the EP-A-0 813 888 and its corresponding U.S. Pat. No. 5,891,170 commonly assigned herewith to ELA Medical, to which one will be able to refer for further details, and which U.S. Pat. No. 5,891,170 is incorporated herein by reference.

Primarily, the further analysis algorithm analyzes a histogram of RR intervals memorized during a given number of cycles, for example, eight cycles. It determines a central peak of stability, and studies whether the percentage of RR intervals located inside the central peak of stability is higher than a given value, for example, 75%. If such is the case, the algorithm determines that there is a stable ventricular rhythm and considers that the detected disorder is a super-rapid VT. In the contrary case, it considers that the rhythm is unstable and is VF.

To be able to differentiate the stimulation therapies that will be applied (ATP or shock), in the event of a VT rhythm, the algorithm increments a counter that counts persistence of fast VT, while leaving unchanged a counter of persistence of VF. In the contrary case, on determination of a VF, the VF and VT persistence counters are both incremented. When the VT persistence counter reaches a given level, an ATP therapy is implemented; if the VF persistence counter is first to reach its given threshold for the release of therapy, then a defibrillation shock is immediately applied to the patient. Any suitable threshold count for the persistence counter can be used, e.g., 12 counts for VT and 6 counts for VF, which numbers are programmable.

Suitable devices for which the present invention has application include, for example, the Defender™ and Alto™ brand of defibrillators available from Ela Médical, Montrouge, France. These devices are microprocessor-based systems having known circuits for receiving, conditioning and processing detected electrical signals, and are capable of receiving software instructions by telemetry, storing them in memory, and then executing those instructions to perform the functions described above in implementing the present invention. The creation of suitable software instructions for controlling an implant to perform the aforementioned functions of the present invention are believed to be within the abilities of a person of ordinary skill in the art. Similarly, the detection circuits used to detect the cardiac signals in the atrium and the ventricle in the left and/or right chambers, as well as the circuits for delivering ATP and shock stimulation therapies of controllable energy levels, are well known, and any suitable design thereof may be used.

One skilled in the art will appreciate that the present invention can be practiced by other than the described embodiments, which are presented for purposes of illustration and not of limitation.

I claim:

1. An active implantable-medical device for delivery of antitachycardia stimulation therapy, comprising:
means for detecting ventricular and atrial cardiac activity and a cardiac rhythm;
means for delivering an antitachycardia therapy by means of a stimulation selected from among the group consisting of a defibrillation shock, a cardioversion shock, and an antitachycardia pacing;
means for discriminating ventricular arrhythmias in response to detected ventricular events comprising means for determining a ventricular frequency, means for comparing the determined ventricular frequency to at least a first threshold of detection of ventricular tachycardia (TDI), and a second threshold of detection of ventricular fibrillation (FDI);
means for inhibiting the application of a said stimulation in response to the determined ventricular frequency being lower than said first threshold (TDI);
means for suspecting a ventricular tachycardia and continuing an analysis of the determined cardiac rhythm with respect to a first set of predetermined criteria in response to the determined ventricular frequency being between the first threshold (TDI) and the second threshold (FDI);
means for commanding an application of a shock therapy in response to the determined ventricular frequency being higher than said second threshold (FDI);
means for providing a third threshold (F/TDI) between the first threshold (TDI) and the second threshold (FDI), wherein the discriminating means further comprises means for suspecting a ventricular tachycardia and continuing the analysis of the cardiac rhythm with respect to the said first set of criteria only in response to the determined ventricular frequency is between the first threshold (TDI) and third threshold (F/TDI), and
means for operating an additional discrimination between a ventricular tachycardia and a ventricular fibrillation in response to the determined ventricular frequency being between the third threshold (F/TDI) and second threshold (FDI).

2. The device of claim 1 wherein the third threshold (F/TDI) is selected from between 190 and 210 bpm and the second threshold (FDI) selected from between 230 and 250 bpm.

3. The device of claim 1 wherein the third threshold (F/TDI) and the second threshold (FDI) are separated by an interval selected from between 20 and 50 bpm.

4. The device of claim 1 wherein the means for operating the additional discrimination further comprises means for determining a stability of the detected ventricular frequency.

5. The device of claim 4, wherein the commanding means in response to said additional discrimination commands an application of a shock therapy in response to the determined ventricular frequency being determined unstable, and commands an application of an antitachycardia pacing in response to the determined ventricular frequency being determined stable.

6. The device of the claim 4 wherein the means for determining said stability further comprises means for performing a statistical analysis of intervals RR.

7. The device of claim 6 wherein said statistical analysis performing means further comprises means for establishing a histogram of intervals RR, searching for a peak of stability, and evaluating a proportion of intervals included in said peak of stability.
